(19) United States
(12) Patent Application Publication
(10) Pub. No.: US 2020/0234820 A1
(43) Pub. Date: Jul. 23, 2020

(54) INFUSION MANAGEMENT METHOD AND SYSTEM

(71) Applicant: MEDCAPTAIN MEDICAL TECHNOLOGY CO., LTD., Shenzhen, Guangdong (CN)

(72) Inventors: Shendou JIANG, Shenzhen, Guangdong (CN); Yazhou TANG, Shenzhen, Guangdong (CN); Jun DONG, Shenzhen, Guangdong (CN)

(21) Appl. No.: 15/758,846

(22) PCT Filed: Feb. 20, 2017
(86) PCT No.: PCT/CN2017/074127

§ 371 Date: Mar. 9, 2018

(30) Foreign Application Priority Data
Feb. 13, 2017 (CN) 201710076506.4

(51) Int. Cl. G16H 40/67; A61M 5/14; G16H 20/17; G16H 10/60; G16H 70/40; G16H 70/20; G16H 40/40; G05B 19/042

(52) U.S. Cl. CPC G16H 40/67; A61M 5/1407; G16H 20/17; G16H 10/60; G16H 70/40; G16H 70/20; G05B 2219/23329; G05B 19/042; A61M 2205/6009; A61M 2205/52; A61M 2205/18; A61M 2205/6072; G16H 40/40

(57) ABSTRACT

An infusion management method and a system are provided. The system includes the following. A central station is configured to acquire first patient information and first medication information, and send the first patient information and the first medication information to a work station. The work station is configured to receive the first patient information and the first medication information and save the first patient information and the first medication information as first information. The central station is further configured to generate a patient information carrier and a medication information carrier corresponding to the first medication information and the first medication information. An information reading device is configured to acquire second information from the patient information carrier and the medication information carrier. The work station is configured to acquire third information from the central station, acquire the second information, and determine whether the information are the same.

### CROSS REFERENCE TO RELATED APPLICATIONS

This application claims priority to Chinese Patent Application No. 201710076506.4, filed on Feb. 13, 2017, and entitled “INFUSION MANAGEMENT METHOD AND SYSTEM”, the disclosure of which is hereby incorporated by reference in its entirety.

### TECHNICAL FIELD

The present disclosure relates to the field of medical technology, and more particularly to an infusion management method and an infusion management system.

### BACKGROUND

With the continuous development of information technology and the ever-increasing demands of people for medical care, medical informatization construction has drawn more and more attention in the industry. At present, one common method is to use a clinical information system (CIS) for medical information management. When performing infusion on a patient, medical staff obtains information from the CIS, and the information including patient information, medical advice information, medication information, and other information, is set and managed through an infusion management system. Information in the infusion management system, such as patient information, medical advice information, medication information, etc., needs to be set manually by medical staff, and then the infusion is performed on a patient according to the information set above. However, in practice, we found that the process of manually setting information is tedious and multiple checks may be required to avoid setting errors, thus leading to longer time to set information, thereby reducing the infusion efficiency.

### SUMMARY

Embodiments of the present disclosure provide an infusion management method and an infusion management system, to improve infusion efficiency.

According to a first aspect of the disclosure, there is provided an infusion management system, which may include a central station and an infusion system. The infusion system may include a work station, an information reading device, and a single pump.

The central station is configured to acquire first patient information and first medication information, and send the first patient information and the first medication information to the work station.

The work station is configured to receive the first patient information and the first medication information from the central station, and save the first patient information and the first medication information as first information.

The central station is further configured to generate information carriers of the first patient information and the first medication information, to obtain a patient information carrier and a medication information carrier.

The information reading device is configured to acquire, according to the first patient information, second information from the patient information carrier and the medication information carrier generated by the central station, and the second information includes second patient information and second medication information.

The work station is further configured to acquire third information from the central station according to the first patient information, and the third information includes third patient information and third medication information.

The work station is further configured to acquire the second information obtained by the information reading device and determine whether the first information, the second information, and the third information are the same. When the first information, the second information, and the third information are the same, the work station is further configured to control the single pump to perform infusion according to the first information.

According to a second aspect of the disclosure, there is provided an infusion management method. The method can be implemented based on an infusion management system including a central station and an infusion system. The infusion management method may include the following.

The central station acquires first patient information and first medication information, and sends the first patient information and the first medication information to a work station of the infusion system.

The work station receives the first patient information and the first medication information from the central station, and saves the first patient information and the first medication information as first information.

The central station generates information carriers of the first patient information and the first medication information to obtain a patient information carrier and a medication information carrier.

An information reading device of the infusion system acquires second information from the patient information carrier and the medication information carrier according to the first patient information, the second information includes second patient information and second medication information.

The work station of the infusion system acquires third information from the central station according to the first patient information, and the third information includes third patient information and third medication information.

The work station acquires the second information obtained by the information reading device and determines whether the first information, the second information, and the third information are the same. When the first information, the second information, and the third information are the same, infusion is performed according to the first information by controlling a single pump of the infusion system.

In embodiments of the present disclosure, the infusion management system includes the central station and the infusion system. The infusion system includes the work station, the information reading device, and the single pump. The central station sends the acquired first information including the first patient information and the first medication information to the work station for infusion. The work station obtains the second information obtained by the information reading device and obtains the third information from the central station. The work station determines whether the first information, the second information, and the third information are the same, and controls the single pump to perform infusion according to the first information when the above information is the same with each other. It can be seen that the work station can automatically acquire the first information for infusion and determine whether the first information is correct, in this way, it is possible to avoid tedious manual setting and repeated checking by the medical staff, and save the time for setting information before infusion, thereby improving the infusion efficiency.

### BRIEF DESCRIPTION OF THE DRAWINGS

To describe the technical solutions in the embodiments of the present disclosure more clearly, the following briefly introduces the accompanying drawings required for describing the embodiments. Apparently, the accompanying drawings in the following description show some embodiments of the present disclosure. Those of ordinary skill in the art may also obtain other drawings based on these accompanying drawings without creative efforts.

FIG. 1 is a schematic structural diagram illustrating an infusion management system according to an embodiment of the present disclosure.

FIG. 2 is a schematic structural diagram illustrating another infusion management system according to an embodiment of the present disclosure.

FIG. 3 illustrates an operation interface of a central station according to an embodiment of the present disclosure.

FIG. 4 illustrates an operation interface of another central station according to an embodiment of the present disclosure.

FIG. 5 is a schematic structural diagram illustrating yet another infusion management system according to an embodiment of the present disclosure.

FIG. 6 is a schematic flow diagram illustrating an infusion management method according to an embodiment of the present disclosure.

FIG. 7 illustrates a flow diagram of a patient infusion process according to an embodiment of the present disclosure.

### DETAILED DESCRIPTION OF ILLUSTRATED EMBODIMENTS

Hereinafter, technical solutions embodied by the embodiments of the disclosure will be described in a clear and comprehensive manner in reference to the accompanying drawings intended for the embodiments. It is evident that the embodiments described herein constitute merely some rather than all of the embodiments of the disclosure, and that those of ordinary skill in the art will be able to derive other embodiments based on these embodiments without making inventive efforts, which all such derived embodiments shall all fall in the protection scope of the disclosure.

Embodiments of the present disclosure provide an infusion management method and an infusion management method system, which can improve infusion efficiency. Detailed description will be given below.

Referring to FIG. 1, FIG. 1 is a schematic structural diagram illustrating an infusion management system according to an embodiment of the present disclosure. As illustrated in FIG. 1, the infusion management system includes a central station 101, and an infusion system 102. The infusion system 102 includes a work station 1021, an information reading device 1022, and a single pump 1023.

The central station 101 is configured to acquire first patient information and first medication information, and send the first patient information and the first medication information to the work station 1021.

In one embodiment, patient information may include one or more of the following information: patient identification (ID), patient name, patient gender, patient age, patient height, patient weight, and other descriptions of the patient. Patient information may further include at least one selected from a group consisting of hospital bed information (designated bed), medical staff information, and so on.

After the patient enters a department, the first patient information and the first medication information can be automatically acquired from a patient information database without manually inputting by the medical staff. For example, the first patient information and the first medication information is retrieved from the patient information database according to the patient information such as the patient ID, and the first medication information may be medication information input by the doctor into the patient information database when diagnosing the patient.

In one embodiment, when there are multiple patients, in order to find target patient information from multiple patient information conveniently, the process of obtaining the patient information and medical advice information corresponding to the patient information may include the following. A search field for finding the patient information input by a user is received; the patient information and the medication information corresponding to the patient information are obtained from the patient information database according to the search field.

As an example, after patient enters a department, medical staff obtains the first patient information by retrieving in the patient information database. For example: Patient ID: 100001, Patient Name: Jack, Patient Gender: Male, Patient Age: 40, Patient Weight: 70, Patient Height: 175, Designated Bed: 03, Medical Staff: Alice, Other Condition Description: None. The patient information can be obtained by entering, in the search dialog box, the patient ID (e.g., 100001) or other search fields such as the patient name, or can also be obtained by searching a patient information list page. Further, the medical staff allows the patient to leave the department when needed, and the patient information can be deleted by patient information management unit.

The work station 1021 is configured to receive the first patient information and the first medication information from the central station 101, and save the first patient information and the first medication information as first information.

In one embodiment, a network connection, such as a wireless connection or a wired connection, may be established between the central station 101 and the work station 1021, for example, a Wi-Fi connection. The work station can automatically obtain the first patient information and the first medication information from the central station without manually inputting by the medical staff. For instance, a button arranged on the central station for sending information to the work station is clicked, and the work station can automatically acquire and then save the first patient information and the first medication information. The work station can include one or more of an infusion pump, an injection pump, and a multi-channel combination pump, and can also include a control portion. The work station can also be one or more of an infusion pump, an injection pump, and a multi-channel combination pump, the work station is applicable to supply power and communicate for the built-in infusion pump and the built-in injection pump.

The central station 101 is further configured to generate information carriers of the first patient information and the first medication information, to obtain a patient information carrier and a medication information carrier.

In one embodiment, the information carrier can be a barcode, a two-dimensional code, or the like that carries the appropriate information. For example, the patient information carrier can be a barcode, a two-dimensional code, or the like that carries the patient information. Information recorded on the patient information carrier may include patient ID, patient name, patient gender, patient age, patient height, patient weight, and other descriptions of the patient. Information recorded on the patient information carrier may further include medical information such as designated bed information, and medical staff information, and infusion information such as infusion medicine code. The patient information carrier can be a barcode or a two-dimensional code generated by the central station, which may be printed and attached to a corresponding patient bed after being generated, or can be a wrist-band with a generated barcode or two-dimensional code for being worn on a patient's wrist. Medical staff information can be used to generate a two-dimensional code or a barcode for the corresponding medical staff to wear, so as to achieve the following verification of the medical staff information.

The patient information and the medication information can be printed on the same information carrier or printed separately; for example, a barcode is used for patient information, and another barcode is used for medication information. The patient information can also be printed barcodes separately. The medication information can include patient basic information such as patient ID to avoid medication errors.

The information reading device 1022 is configured to acquire second information from the patient information carrier and the medication information carrier generated by the central station 101 according to the first patient information, and the second information includes second patient information and second medication information.

In one embodiment, the information reading device can be a radio-frequency identification (RFID) reader, a barcode scanner, or other information reading equipment. The information reading device is configured to read out information corresponding to information carrier, such as the barcode or the two-dimensional code before the single pump performs infusion, to confirm that information in the work station and information carried by the information carrier are the same, and then infusion is performed according to confirmed, and accurate patient information and medication information.

The work station 1021 is further configured to acquire third information from the central station 101 according to the first patient information, and the third information includes third patient information and third medication information.

In one embodiment, the work station 1021 may obtain the third information from the central station 101 through the established network connection, such as a wireless connection (for example, a Wi-Fi connection) or a wired connection. The third information includes the third patient information and the third medication information.

The work station 1021 is further configured to acquire the second information obtained by the information reading device 1022 and determine whether the first information, the second information, and the third information are the same. When the first information, the second information, and the third information are the same, the work station 1021 is configured to control the single pump 1023 to perform infusion according to the first information.

In one embodiment, the purpose of obtaining the third information is to avoid the infusion error. When medical staff corrects the infusion information before or during infusion, infusion information in the central station has been updated, while infusion information in the work station is not updated, thereby information in the work station and information in the central station need to be compared. If information in the work station and information in the central station are the same, indicating that infusion information is the latest, and there is no change at the central station, therefore, infusion can be performed according to the infusion information in the work station. The work station and the information reading device can establish a wired connection or other connections. The single pump 1023 may include at least one of an infusion pump, an injection pump, and a work station.

Additionally, in order to obtain the latest information in the central station, under the condition that the central station has established a connection with the work station, the work station can obtain the third information in the central station with a preset time period, and can also acquire the third information in the central station regularly.

The infusion management system illustrated in FIG. 1 includes the central station and the infusion system. The infusion system includes the work station, the information reading device, and the single pump. The central station sends the acquired first information including the first patient information and the first medication information to the work station for infusion. The work station acquires the second information obtained by the information reading device and acquires the third information from the central station. Continuing, the work station determines whether the first information, the second information, and the third information are the same, and controls the single pump to perform infusion according to the first information when the above information is the same with each other. It can be seen that the work station can automatically acquire the first information for infusion and determine whether the first information is correct, which can avoid tedious manual setting and repeated checking by the medical staff, and save the time for setting information before infusion, thereby improving the infusion efficiency.

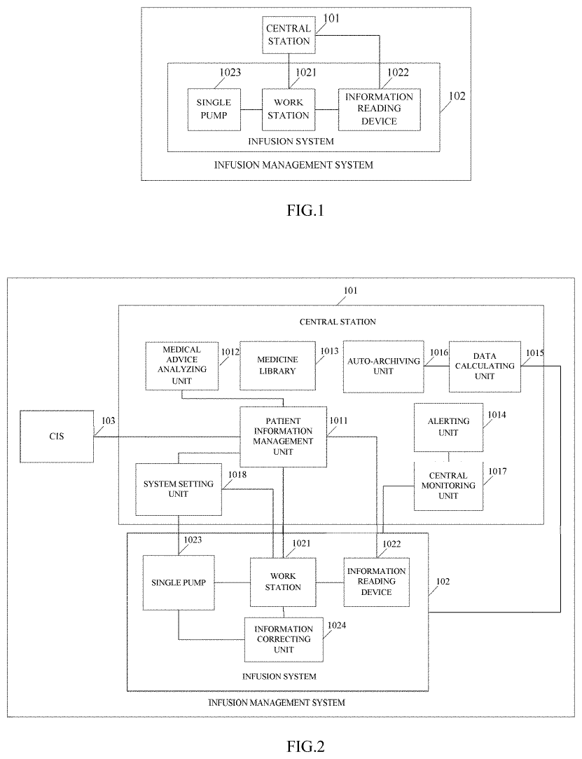

Referring to FIG. 2, FIG. 2 is a schematic structural diagram illustrating another infusion management system according to an embodiment of the present disclosure. FIG. 2 is optimized based on the infusion management system illustrated in FIG. 1. The central station 101 may include a patient information management unit 1011, a medical advice analyzing unit 1012, and a medicine library 1013.

The central station 101 is configured to acquire the first patient information and the first medication information as follows.

The patient information management unit 1011 is configured to acquire the first patient information and the medical advice information corresponding to the first patient information.

The medical advice analyzing unit 1012 is configured to determine the first medication information according to the first patient information and the medical advice information acquired by the patient information management unit 1011 and the medication information in the medicine library 1013.

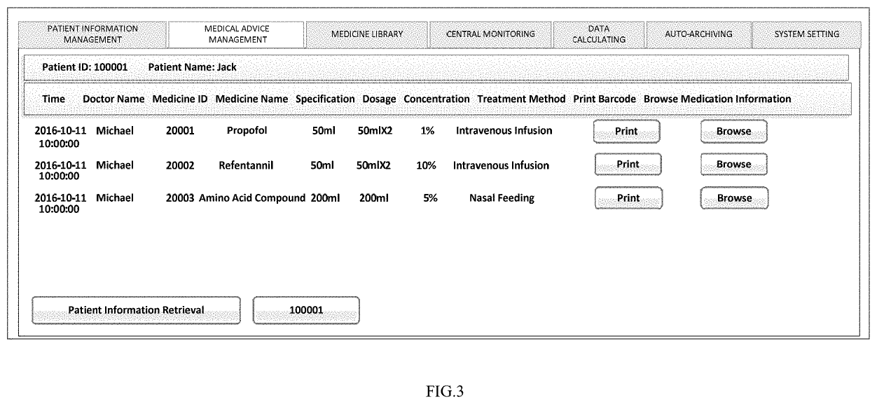

In one embodiment, FIG. 3 illustrates an operation interface of a central station. The medical advice analyzing unit 1012 is configured to automatically obtain medical advice of a patient from the CIS according to the first patient information acquired by the patient information management unit 1011. The infusion information of medicines to be infused through the infusion pump and the injection pump can be automatically confirmed according to the “treatment methods” or the intelligent comparison between the medicines and the medicines in the existing medicine library of the central station.

As an alternative embodiment, the infusion management system can further include a clinical information system (CIS) 103.

The clinical information system 103 is configured to send the first patient information and the medical advice information corresponding to the first patient information.

The patient information management unit 1011 is configured to acquire the first patient information and the medical advice information corresponding to the first patient information as follows.

The patient information management unit 1011 is configured to receive the first patient information and the medical advice information corresponding to the first patient information from the clinical information system 103 are received.

In one embodiment, as illustrated in FIG. 3, a patient information list may be obtained by the central station 101 from the clinical information system 103. The central station can establish a network connection with the CIS, such as a wireless connection or a wired connection, and obtain the patient information and the medical advice information corresponding to the patient information from the CIS through the network connection. The designated bed information, medical staff information, and other medical information can be set in the CIS, and the central station can directly acquire the medical information from the CIS. The medical information can also be set in the central station. As illustrated in FIG. 3, a patient information list page can also be displayed in the central station, to display patient information and other information in the form of a list. Moreover, the patient information can be displayed according to a preset rule; for example, the patient information is displayed according to the alphabetical order of the patients' name.

As an alternative embodiment, the work station 1021 is further configured to determine whether has established a connection with the central station 101. If yes, the manner in which the work station 1021 acquires the third information from the central station 101 according to the first patient information includes the following.

The work station 1021 is configured to obtain the third information from the central station 101 through the connection according to the first patient information.

In particular, the work station 1021 is configured to acquire the third information from the patient information management unit 1011 through the connection according to the first patient information.

As an alternative embodiment, the infusion system 102 may further include an information correcting unit 1024.

The work station 1021 is further configured to determine whether the first information and the second information are the same when the connection between the work station 1021 and the central station 101 has not been established, and control a single pump 1023 to perform infusion according to the first information when the first information and the second information are the same.

The work station 1021 is further configured to send a control command to the information correcting unit 1024 when the first information and the second information are different.

The information correcting unit 1024 is configured to receive, when the control command is received, a correction instruction input by a user for correcting the first information or the second information, and execute the correction instruction to obtain fourth information, where the fourth information includes fourth patient information and fourth medication information.

The single pump 1023 is further configured to perform infusion according to the fourth information obtained by the information correcting unit 1024.

In one embodiment, when the work station cannot be connected to the central station through the network, information carrier which includes the following can be read to obtain patient information. 1) An information carrier that identifies the operator ID, such as a barcode pasted into the work card; 2) information that identifies the patent ID, such as a wrist-band barcode attached to a patient's hand; 3) information identifying the bed ID, such as a barcode affixed to the bedside; 4) barcode information identifying patient information, medication information, and the like. The work station is configured to determine: 1) whether barcode information read out from the patient and medication information barcode is the same as barcode information identifying the operator, barcode information identifying the bed, and barcode information identifying the patient ID; 2) whether information from the patient and the medication information barcode is the same as information in an infusion pump and an injection pump, and information built-in in the work station; the above information will be automatically input when the information is consistent; the above parameters will be manually corrected when the information is inconsistent, the corrected parameters will be received by the information correcting unit 1024, and the single pump 1023 will be invoked for infusion.

As an alternative embodiment, the central station 101 may further include an alerting unit 1014.

The alerting unit 1014 is configured to generate and output an alerting signal for prompting a user when the infusion system satisfies a fault condition in a fault database.

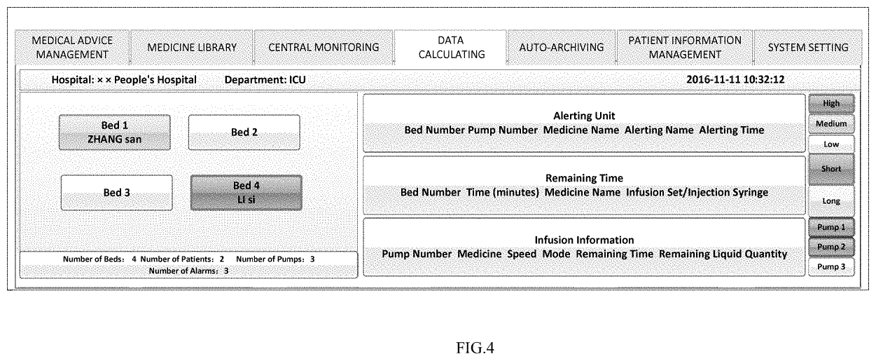

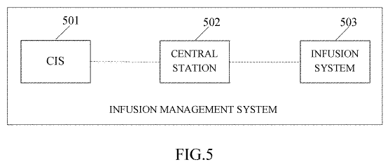

In one embodiment, FIG. 4 illustrates an operation interface of another central station. As illustrated in FIG. 4, a central monitoring unit is configured to monitor various states, such as an infusion state, an alerting state, remaining time, and so on, to achieve remote safety monitoring. When an abnormal condition meets the fault condition in the fault database, the corresponding alerting information, such as bed number and single pump number, medicine name, as well as the alerting name and the alerting time, is displayed.

As an alternative embodiment, the central station 101 may further include a data calculating unit 1015 and an auto-archiving unit 1016.

The data calculating unit 1015 is configured to acquire, from the infusion system 102, infusion state parameters corresponding to the first patient information.

The auto-archiving unit 1016 is configured to save the first patient information, the first medication information and the infusion state parameters acquired by the data calculating unit 1015.

In one embodiment, the central station 101 may further include the data calculating unit 1015 and the auto-archiving unit 1016. As illustrated in FIG. 4, the infusion state parameters may include infusion data during the infusion process, such as the total input amount of a patient per unit time, the input amount of a certain medicine in unit time, etc., and may further include equipment parameters such as infusion pump number. The central station and the work station can establish a connection, such as a network connection including a wireless and wired network connection. The central station can obtain infusion state parameters corresponding to the patient information from the work station in a preset time interval and store them in an infusion state parameter list. The central station can calculate the infusion data during the entire infusion process according to the infusion status parameter list, and these infusion data can be summarized as the infusion state parameters.

The auto-archiving unit 1016 is configured to automatically store all infusion data of the infusion process; the infusion data including the patient information, the medication information, and state data of the infusion process is stored in a data format, such as a PDF, to a backup data area or backup server.

As an alternative embodiment, the central station 101 may further include a central monitoring unit 1017.

The central monitoring unit 1017 is configured to monitor an infusion state of the infusion system 102.

When the infusion system 102 satisfies the fault condition in the fault database, the manner in which the alerting unit 1014 generates and outputs the alerting signal for prompting the user includes the following.

When the infusion state monitored by the central monitoring unit 1017 satisfies the fault condition in the fault database, the alerting signal is output.

In one embodiment, as illustrated in FIG. 4, the central monitoring unit 1017 can monitor the designated beds for infusion corresponding to all patient information in the central station so as to notify the medical staff to solve in time when abnormalities occur. When the connection between the central station and the work station has not been established, the fault condition can be an abnormal operation of the central station, for example, a system problem that the information cannot be saved, or other problems. However, when the connection between the central station and the work station has been established, the central station can monitor the working state of the work station in real time, an alerting signal is output when an abnormal occurs in the work station. For instance, an alerting signal is output when an infusion on one bed has been blocked, the corresponding infusion device has been damaged, or one infusion has been completed, or that patient information or infusion information has been changed in the work station or in the central station is detected. Moreover, in order to make the alerting signal more targeted, that is, the medical staff can clearly understand the cause of the abnormal situation at a glance, for different faults in the fault database; each fault can be provided with one alerting signal, and different alerting signals are used to prompt the different faults. The alerting signal can be a voice output, a text display output, or an output in other prompt modes, the present disclosure is not limited thereto.

As an alternative embodiment, the central station 101 may further include a system setting unit 1018.

The system setting unit 1018 is configured to set system parameters for the infusion management system.

In one embodiment, the system setting unit 1018 is configured to set a connection network address of the CIS, network connection addresses of the work station and the single pump, number of beds, administrator authorities, languages, time, and other settings. The system setting unit 1018 can at least establish a connection with a work station 1021, a single pump 1023, and a patient information management unit 1011.

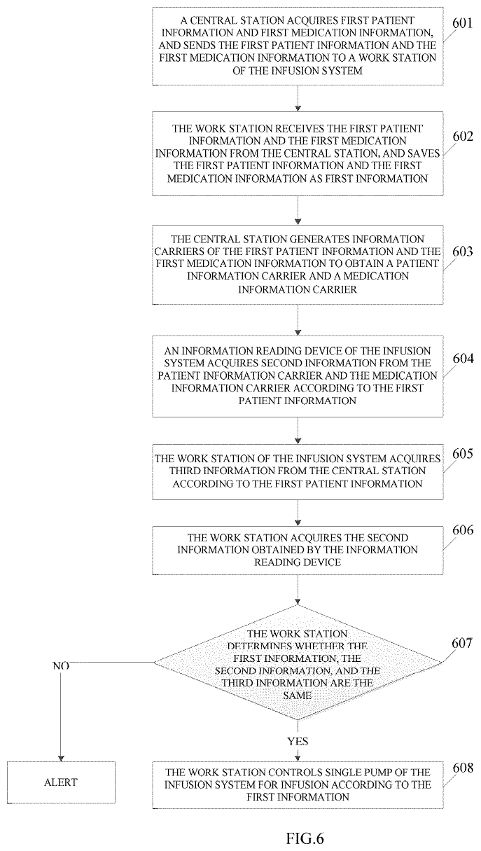

Referring to FIG. 5, FIG. 5 is a schematic structural diagram illustrating yet another infusion management system according to an embodiment of the present disclosure. The infusion management system may at least include a central station 501 and an infusion system 502. A dotted line between the central station 501 and the work station 502 indicates that the two can be connected wirelessly or wired. The structure of the central station 501 can be the same as that of the central station illustrated in FIG. 1 and FIG. 2, the structure of the work station 502 can be the same as that of the central station illustrated in FIG. 1 and FIG. 2, and this disclosure will not describe repeatedly again. In an alternative embodiment, as illustrated in FIG. 5, the infusion management system further includes a clinical information system 503, and a dotted line between the clinical information system 503 and the central station 501 indicates a wireless connection or a wired connection.

In the infusion management system illustrated in FIG. 2, the infusion management system includes the central station and the infusion system, and the infusion system includes the work station, the information reading device, and the single pump. The first information including the first patient information and the first medication information acquired by the central station are sent to the work station for infusion. The work station is configured to acquire the second information obtained by the information reading device and acquire the third information from the central station. Continuing, the work station is configured to determine whether the first information, the second information, and the third information are the same, and if yes, control the single pump to perform infusion according to the first information. Therefore, the work station can automatically acquire the first information for infusion and determine whether the first information is correct, which can avoid the cumbersome manual setting and repeated check by the medical staff, therefore the time for setting information before infusion is saved, thereby improving the infusion efficiency.

Referring to FIG. 6, FIG. 6 is a schematic flow diagram illustrating an infusion management method according to an embodiment of the present disclosure. As illustrated in FIG. 6, the infusion management method may include the following operations.

At 601, a central station acquires first patient information and first medication information, and sends the first patient information and the first medication information to a work station of the infusion system.

As an alternative embodiment, the central station acquires the first patient information and the first medication information as follows.

A patient information management unit of the central station acquires the first patient information and medical advice information corresponding to the first patient information.

A medical advice analyzing unit of the central station determines the first medication information according to the first patient information and the medical advice information acquired by the patient information management unit and a medicine library.

As an alternative embodiment, the infusion management system may further include a CIS, and the infusion management method may further include the following.

The CIS sends the first patient information and the medical advice information corresponding to the first patient information.

The patient information management unit acquires the first patient information and the medical advice information corresponding to the first patient information as follows.

The patient information management unit receives the first patient information and the medical advice information corresponding to the first patient information from the CIS.

At 602, the work station receives the first patient information and the first medication information from the central station, and saves the first patient information and the first medication information as first information.

At 603, the central station generates information carriers of the first patient information and the first medication information to obtain a patient information carrier and a medication information carrier.

At 604, an information reading device of the infusion system acquires second information from the patient information carrier and the medication information carrier according to the first patient information, and the second information includes second patient information and second medication information.

At 605, the work station of the infusion system acquires third information from the central station according to the first patient information, and the third information includes third patient information and third medication information.

As an alternative embodiment, before the work station acquires the third information from the central station according to the first patient information, the infusion management method may further include the following.

The work station determines whether has established a connection with the central station. When the work station has established a connection with the central station, the work station can acquire the third information from the central station according to the first patient information as follows.

The work station acquires the third information from the central station through the connection according to the first patient information.

As an alternative embodiment, when the work station has not established a connection with the central station, the infusion management method may further include the following.

The work station determines whether the first information and the second information are the same, and triggers the operation of controlling a single pump of the infusion system to perform infusion according to the first information.

When the first information and the second information are different, a correction instruction input by a user for correcting the first information or the second information is received by an information correcting unit of the infusion system, and the correction instruction is executed to obtain fourth information, the fourth information includes fourth patient information and fourth medication information.

The single pump performs infusion according to the fourth information.

At 606, the work station acquires the second information obtained by the information reading device.

At 607, the work station determines whether the first information, the second information, and the third information are the same. If yes, proceed to step 608; otherwise, proceed to alerting.

At 608, the work station controls the single pump of the infusion system for infusion according to the first information.

As an alternative embodiment, the central station may further include an alerting unit, and the infusion management method can include the following.

When the infusion system satisfies a fault condition in a fault database, the alerting unit generates and outputs an alerting signal for prompting a user.

As an alternative embodiment, the infusion management method can further include the following.

A data calculating unit of the central station acquires, from the infusion system, infusion state parameters corresponding to the first patient information.

An auto-archiving unit of the central station saves the first patient information, the first medication information, and the infusion state parameters.

As an alternative embodiment, the infusion management method may further include the following.

A central monitoring unit of the central station monitors an infusion state of the infusion system.

When the infusion system satisfies the fault condition in the fault database, the alerting unit generates and outputs the alerting signal for prompting the user as follows.

When the infusion state monitored by the central monitoring unit satisfies the fault condition in the fault database, the alerting signal is output.

As an alternative embodiment, the infusion management method may further include the following.

System parameters of the infusion management system are set by a system setting unit of the central station.

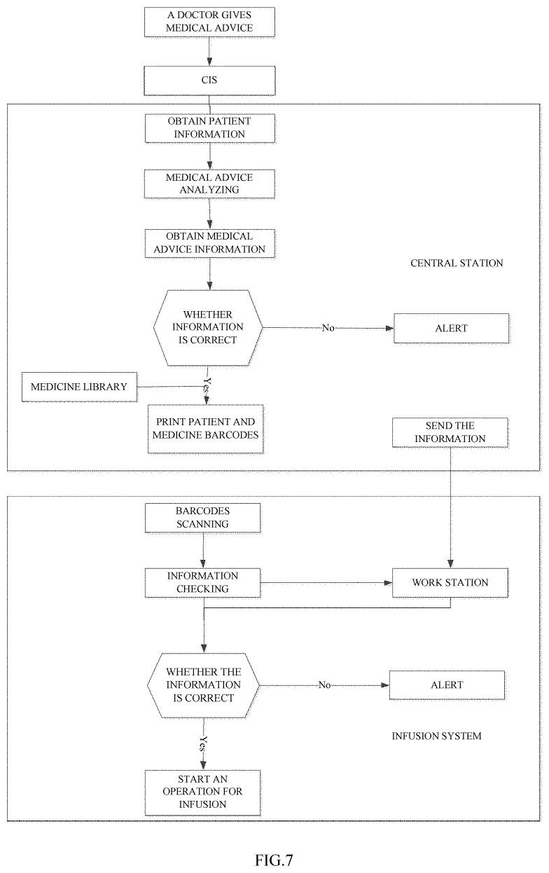

In one embodiment, as illustrated in FIG. 7, FIG. 7 illustrates a flow diagram of a patient infusion process, which is realized on the basis of the infusion management method illustrated in FIG. 6. First, a doctor gives medical advice to a patient and enters medical advice information in the CIS. The medical advice information may include medications for treatment and an artificial nursing method. Second, after the patient enters the department, medical staff of the department assigns the designated beds and designated medical staff who are allowed to operate. Third, medical staff obtain all patient information in the central station from the CIS according to the patient ID number or other patient search fields, and the patient information may include the name, sex, age, weight, and other parameters. The medical advice analyzing unit of the central station automatically analyzes infusion medication information required by an infusion pump and an injection pump from the CIS based on the patient ID, and the medication information includes categories and doses of medicines such as Propofol and Refentannil. Fourth, whether the above information obtained from the CIS is correct should be confirmed by the medical staff. If yes, the patient and medicine barcodes are printed, which may include all the information of a patient, bed information, permitted operator information, key infusion information of the medicine, such as the name and dose of the medicine. The medical staff prepares the medicine and pastes the barcode into the infusion bag or injector. Fifth, if the infusion system can be connected to the central station, the central station sends the patient information and medication information to the infusion system. Sixth, after the medical staff prepares the medicine and pastes into the barcode and before infusion, perform: 1) scanning a barcode identifying the operator ID; 2) scanning a barcode such as a wrist-band barcode identifying a patient ID; 3) scanning an ID barcode identifying a bed number; 4) scanning the patient information barcode and the medication information barcode.

In case that the infusion system is connected to the central station via a network connection device, a) the infusion system can automatically determine whether the above four barcode information, the stored patient information and the medication information, as well as the patient information and the medication information in the central station are the same; the infusion system can automatically determine whether the medication information and the built-in medicine library information are consistent; if yes, all the infusion information will be automatically entered, otherwise, the system will alarm to prompt that the information is abnormal.

In case that the work station, the infusion pump or the injection pump are not connected to the central station via a network connection device: a) the work station, the infusion pump or the injection pump can automatically determine whether the above four barcode information are the same; b) the work station, the infusion pump, and the injection pump can automatically determine whether the medication information and the built-in medicine library information are consistent; if yes, all the infusion information will be automatically entered, otherwise, the above information will be manually corrected and the above parameters will be automatically input.

Seventhly, medical staff may start an operation for infusion. After infusion started, in case that the work station, the infusion pump or the injection pump are connected to the central station via a network connection device, the central station can monitor the state of the infusion pump. For instance, when the work station, infusion pump or injection pump are abnormal, or the infusion state is abnormal, the central station alarms on the corresponding bed and prompts the corresponding alerting information; for instance, infusion is blocked, infusion has been completed and other alerting information. The central station regularly and automatically archives all infusion information including the medication to be used by a patient, a serial number of the infusion pump used, infusion parameters, the infusion state of the infusion pump, and other information.

In the infusion management method illustrated in FIG. 6, the infusion management system includes the central station and the infusion system, and the infusion system includes the work station, the information reading device, and the single pump. The central station sends the acquired first information including the first patient information and the first medication information to the work station for infusion. The work station acquires the second information obtained by the information reading device and obtains the third information from the central station. The work station further determines whether the first information, the second information, and the third information are the same, when the above information is the same with each other, the work station will control the single pump for infusion according to the first information. It can be seen that the work station can automatically acquire the first information for infusion and determine whether the first information is correct, thereby avoiding the cumbersome manual setting and repeated check of the medical staff, saving the time for setting the information before the infusion and improving the infusion efficiency.

The infusion management method and a system disclosed in the embodiments of the present disclosure are described in detail above. Some examples are used herein to describe the principle and implementation of the present disclosure. The description of the foregoing embodiments is merely used to help understand the method and its core idea of the present disclosure. Meanwhile, for those skilled in the art, according to the idea of the present disclosure, there are some changes in the specific implementation manners and the application scope. To sum up, the content of this description should not be construed as a limitation to the present disclosure.

What is claimed is:

1. An infusion management system, comprising:
a central station; and
an infusion system, comprising a work station, an information reading device, and a single pump; wherein
the central station is configured to acquire first patient information and first medication information, and send the first patient information and the first medication information to the work station;
the work station is configured to receive the first patient information and the first medication information from the central station, and save the first patient information and the first medication information as first information;
the central station is further configured to generate information carriers of the first patient information and the first medication information, to obtain a patient information carrier and a medication information carrier;
the information reading device is configured to acquire, according to the first patient information, second information from the patient information carrier and the medication information carrier generated by the central station, wherein the second information comprises second patient information and second medication information;
the work station is further configured to acquire third information from the central station according to the first patient information, wherein the third information comprises third patient information and third medication information; and
the work station is further configured to acquire the second information obtained by the information reading device, determine whether the first information, the second information, and the third information are the same, and control the single pump to perform infusion according to the first information when the first information, the second information, and the third information are the same.

2. The system of claim 1, wherein the central station comprises a patient information management unit, a medical advice analyzing unit, and a medicine library;
the manner in which the central station acquires the first patient information and the first medication information comprising:
the patient information management unit being configured to acquire the first patient information and medical advice information corresponding to the first patient information; and
the medical advice analyzing unit being configured to determine the first medication information according to the first patient information and the medical advice information acquired by the patient information management unit and medication information in the medicine library.

3. The system of claim 2, the infusion management system further comprising a clinical information system (CIS), wherein
the CIS is configured to send the first patient information and the medical advice information corresponding to the first patient information; and
the manner in which the patient information management unit acquires the first patient information and the medical advice information corresponding to the first patient information comprises:
the patient information management unit being configured to receive the first patient information and the medical advice information corresponding to the first patient information from the CIS.

4. The system of claim 2, wherein the work station is further configured to determine whether a connection has been established with the central station; and
the manner in which the work station acquires the third information from the central station according to the first patient information when the work station has established the connection with the central station comprising:
the work station being configured to acquire the third information from the central station through the connection according to the first patient information.

5. The system of claim 4, the infusion system further comprising an information correcting unit, wherein
the work station is further configured to determine whether the first information and the second information are the same when the connection between the work station and the central station has not been established, and control the single pump to perform infusion according to the first information when the first information and the second information are the same;
the work station is further configured to send a control command to the information correcting unit when the first information and the second information are different;
the information correcting unit is configured to receive, when the control command is received, a correction instruction input by a user for correcting the first information or the second information, and execute the correction instruction to obtain fourth information, wherein the fourth information comprises fourth patient information and fourth medication information; and
the single pump is further configured to perform infusion according to the fourth information obtained by the information correcting unit.

6. The system of claim 2, the central station further comprising an alerting unit,
wherein the alerting unit is configured to generate and output an alerting signal for prompting a user when the infusion system satisfies a fault condition in a fault database.

7. The system of claim 2, the central station further comprising a data calculating unit and an auto-archiving unit, wherein
the data calculating unit is configured to acquire, from the infusion system, infusion state parameters corresponding to the first patient information; and
the auto-archiving unit is configured to save the first patient information, the first medication information, and the infusion state parameters acquired by the data calculating unit.

8. The system of claim 6, the central station further comprising a central monitoring unit, wherein
the central monitoring unit is configured to monitor an infusion state of the infusion system; and
the manner in which the alerting unit generates and outputs the alerting signal for prompting the user when the infusion system satisfies the fault condition in the fault database comprises:
the alerting unit being configured to output the alerting signal when the infusion state monitored by the central monitoring unit satisfies the fault condition in the fault database.

9. The system of claim 2, the central station further comprising a system setting unit, wherein the system setting unit is configured to set system parameters for the infusion management system.

10. An infusion management method, being implemented based on an infusion management system comprising a central station and an infusion system, and comprising:
acquiring, by the central station, first patient information and first medication information and sending the first patient information and the first medication information to a work station of the infusion system;
receiving, by the work station, the first patient information and the first medication information from the central station and saving the first patient information and the first medication information as first information;
generating, by the central station, information carriers of the first patient information and the first medication information to obtain a patient information carrier and a medication information carrier;
acquiring, by an information reading device of the infusion system, second information from the patient information carrier and the medication information carrier according to the first patient information, the second information comprising second patient information and second medication information;
acquiring, by the work station of the infusion system, third information from the central station according to the first patient information, the third information comprising third patient information and third medication information; and
acquiring, by the work station, the second information obtained by the information reading device, determining whether the first information, the second information, and the third information are the same, and controlling a single pump of the infusion system to perform infusion according to the first information when the first information, the second information, and the third information are the same.

11. The method of claim 10, wherein acquiring first patient information and first medication information by the central station comprises:
acquiring, by a patient information management unit of the central station, the first patient information and medical advice information corresponding to the first patient information; and
determining, by a medical advice analyzing unit of the central station, the first medication information, according to the first patient information and the medical advice information acquired by the patient information management unit and a medicine library.

12. The method of claim 11, wherein the infusion management system further comprises a clinical information system (CIS), and the infusion management method further comprises:
sending, by the CIS, the first patient information and the medical advice information corresponding to the first patient information; and
acquiring the first patient information and medical advice information corresponding to the first patient information by a patient information management unit comprising:
receiving, by the patient information management unit, the first patient information and the medical advice information corresponding to the first patient information from the CIS.

13. The method of claim 11, further comprising the following before the work station acquires third information from the central station according to the first patient information:
determining, by the work station, whether has established a connection with the central station; and
acquiring third information from the central station according to the first patient information by the work station when the work station has established the connection with the central station comprising:
acquiring, by the work station, the third information from the central station through the connection according to the first patient information.

14. The method of claim 13, further comprising the following when the connection between the work station and the central station has not been established:
determining, by the work station, whether the first information and the second information are the same, and triggering the process of controlling the single pump of the infusion system to perform infusion according to the first information when the first information and the second information are the same;
receiving, by an information correcting unit, a correction instruction input by a user to correct the first information or the second information when the first information and the second information are different, and executing the correction instruction to obtain fourth information, the fourth information comprising fourth patient information and fourth medication information; and
performing, by the single pump, infusion according to the fourth information.

15. The method of claim 11, wherein the central station further comprises an alerting unit, and the method further comprises:
generating and outputting, by the alerting unit, an alerting signal for prompting a user, when the infusion system satisfies a fault condition in a fault database.

16. The method of claim 11, further comprising:
acquiring from the infusion system, by a data calculating unit of the central station, infusion state parameters corresponding to the first patient information; and
saving, by an auto-archiving unit of the central station, the first patient information, the first medication information, and the infusion state parameters.

17. The method of claim 15, wherein the infusion management method further comprises:
monitoring, by a central monitoring unit of the central station, an infusion state of the infusion system; and
generating and outputting an alerting signal for prompting a user by the alerting unit, when the infusion system satisfies a fault condition in a fault database comprising:
outputting, by the alerting unit, the alerting signal when the infusion state monitored by the central monitoring unit satisfies the fault condition in the fault database.

18. The method of claim 11, further comprising:
setting, by a system setting unit of the central station, system parameters for the infusion management system.

19. The system of claim 3, the central station further comprising a data calculating unit and an auto-archiving unit, wherein
the data calculating unit is configured to acquire, from the infusion system, infusion state parameters corresponding to the first patient information; and
the auto-archiving unit is configured to save the first patient information, the first medication information, and the infusion state parameters acquired by the data calculating unit.

20. The system of claim 4, the central station further comprising a data calculating unit and an auto-archiving unit, wherein
the data calculating unit is configured to acquire, from the infusion system, infusion state parameters corresponding to the first patient information; and
the auto-archiving unit is configured to save the first patient information, the first medication information, and the infusion state parameters acquired by the data calculating unit.
